(19) United States
(12) Patent Application Publication
(10) Pub. No.: US 2011/0282259 A1
(43) Pub. Date: Nov. 17, 2011

(54) Methods of manufacture of a diffusion dressing

(76) Inventors: Olivier B. Postel, Redwood City, CA (US); Amie B. Franklin, Las Vegas, NV (US)

(21) Appl. No.: 12/719,609

(22) Filed: Mar. 8, 2010

Related U.S. Application Data
(60) Provisional application No. 61/158,460, filed on Mar. 9, 2009.

(51) Int. Cl. A61F 13/02; B32B 38/10; A61F 13/15; A61L 15/22; A61L 15/44

(52) U.S. Cl. 602/46; 602/54; 602/48; 604/378; 156/256

(57) ABSTRACT

A method for manufacturing a diffusion dressing includes automated steps for pre-forming and precutting a first occlusive layer and precutting a permeable layer of material to a finished size, coating one side of the permeable layer with a permeable adhesive, placing precut sections of a cellular material layer onto the adhesive side of the permeable layer, sealing the first occlusive layer to the permeable layer, precutting a section of absorbent material to a finished size, coating the remaining side of the permeable layer with a permeable adhesive, placing the section of absorbent material on the coated side of the permeable layer, applying a seal about the periphery of the remaining side of the absorbent material, pre-forming and precutting an adhesion layer to a finished size, precutting a removable second occlusive layer of material to a finished size, and sealing the second occlusive layer against the adhesion layer.

### CROSS-REFERENCE TO RELATED APPLICATIONS

The present application claims priority to Provisional patent application No. 61/158,460, filed Mar. 9, 2009, the disclosure of which is included herein in its entirety at least by reference.

### BACKGROUND OF THE INVENTION

1. Field of the Invention

The present invention is in the field of medical technologies and more particularly the treatment of tissue via a diffusion type dressing, the invention pertaining to manufacture of such dressings and to dressings providing multiple therapies.

2. Discussion of the State of the Art

In the medical field is often necessary to use devices such as wound and tissue dressings to cover and protect during treatment. Oxygen gas has been shown to provide an added benefit to wound and tissue healing as well as to cell survival in tissue regeneration. Providing gasses such as oxygen through therapeutic diffusion devices to the target area such wounds tissue and cells for an extended period is necessary for treatment and survival. Therapeutic devices such as diffusion dressings may be used to treat such tissue by delivering healing agents and protecting the tissue from infection and the like.

A diffusion dressing is a multi-layered dressing or device that has a reservoir containing a therapeutic agent such as a gas or the like diffused to the tissue or cells in a target area from the dressing interfacing with the fluid, tissue or cells.

It is desirable in the art that therapeutic diffusion devices such as dressings could be manufactured efficiently using automated equipment with state-of-art features however to date conventional means for automated manufacturing of therapeutic gas diffusion devices are unknown.

What is clearly needed are better and more reliable methods of manufacturing such devices such as dressings and what is further needed are dressings that can be regulated relative to the diffusion of therapeutic agents and that can deliver more than one type of agent to tissue or cells being treated.

### SUMMARY OF THE INVENTION

The problem stated above is that while it is desirable that diffusion dressings may be manufactured efficiently using automated equipment with state-of-art features, conventional means for manufacturing diffusion dressings are not efficient for wholesale manufacturing.

The inventor therefore considered automation components and processes looking for elements and sub-processes that exhibited interoperability and full automation capabilities that could potentially be harnessed and integrated to provide a manufacturing process for a diffusion dressing that was efficient, automated, and repeatable.

Every diffusion dressing depends on a combination of specific material layers having specific qualities that working together enable efficient delivery of therapeutic agent to a wound being treated by the dressing. The specific layer compositions and different ways to attach them together around a source of therapeutic agent are typically a part of such apparatus used in making a diffusion dressing.

The inventors realized in an intuitive moment that if an automated manufacturing process could be realized for manufacturing diffusion dressings having rather complex architectures, significant reduction in work and production delays might result. Therefore, the inventors created a unique manufacturing process for diffusion dressings that allowed full automation and repeatability in terms of quality. A significant reduction in manual processing resulted with no impediment to quality or architectural variation created.

Accordingly, in an embodiment of the present invention, a method is provided for manufacturing a diffusion dressing comprising the steps, (a) pre-forming and precutting a first occlusive layer of material to a finished size, (b) precutting a permeable layer of material to a finished size, (c) coating one side of the permeable layer of step (b) with a permeable adhesive, (d) placing one or more precut sections of a cellular material layer onto the adhesive side of the permeable layer of step (b), (e) sealing the first occlusive layer of step (a) to the permeable layer of step (b) encapsulating the one or more sections of cellular material of step (d), (f) precutting a section of absorbent material to a finished size, (g) coating the remaining side of the permeable layer of step (b) with a permeable adhesive, (h) placing the section of absorbent material on the coated side of the permeable layer of step (g), (i) applying a seal about the periphery of the remaining side of the absorbent material forming a peripheral seal to the occlusive layer, (j) pre-forming and precutting a breathable adhesion layer to a finished size sufficient to cover at least the peripheral area of the diffusion dressing thus far assembled, (k) precutting a removable second occlusive layer of material to a finished size, and (l) sealing the second occlusive layer against the adhesion layer of step (j).

In one aspect of the method in step (a), and in step (j), pre-forming is accomplished using a cold vacuum or hot vacuum forming tool. In one aspect of the method in steps (a), (b), (f), (j), and (k), precutting is accomplished using a flatbed, rotary, or hybrid die cutting tool. In a preferred aspect of the method in step (d), and in step (h), the material sections are placed via an island placement technique. Also in a preferred aspect in step (a), and step (k), the occlusive layers are continuous hermetic quality films.

In one aspect in step (d), the cellular material is foam having an open cell structure. In one aspect in step (f), the absorbent material is a hydrocolloid, and or one of open cell polyurethane foam, non-woven rayon, blown microfibers, absorbent beads, adhesive material or gel or blown microfibers. In one aspect in steps (e), (i), and (l), sealing is accomplished using one of heat sealing, radio frequency (RF) cold welding, electron beam welding, or pressure sensitive adhesive. In one aspect of the method in step (i), sealing is accomplished using a double-sided occlusive tape or by laying a bead of impermeable gel.

According to another aspect of the method, an additional step is provided between step (h) and step (i) for attaching a non-adhesive film to the remaining side of the absorbent layer to prevent tissue from growing into the absorbent layer. In still another aspect, an additional step is provided between step (h) and step (i) for applying a gel, a cream, or any other compound that is bio-absorbable or bio-degradable. In this aspect, the bio compound is inert or contains one or more drugs such as an anesthetic, an analgesic or a combination of therapeutic agents.

According to one embodiment of the present invention, a diffusion dressing is provided and includes two or more discrete sections of cellular material enclosed by an occlusive material layer and a permeable material layer and a sealable adhesive interface for maintaining the dressing in a state of adhesion against a tissue area being treated.

In one embodiment, the cellular material is foam having an open cell structure. In one embodiment, the two or more discrete sections of cellular material are charged with a same therapeutic agent. In another embodiment, the two or more discrete sections of cellular material are individually charged with different therapeutic agents. In a variation of this embodiment, the two or more discrete sections of cellular material have different rates of diffusion.

In one embodiment wherein there are multiple cellular sections, the rate of diffusion of each section of material is controlled by controlling the thickness of and or number of layers of permeable adhesive added to the diffusion membrane directly beneath a cellular material section. In one embodiment, the sealable interface comprises an adhesion layer and a second occlusive layer. In a variation of this embodiment, the adhesion layer is a breathable skin adhesive layer and the second occlusive layer is a removable liner that is removed before application of the diffusion dressing. In another variation of this embodiment the adhesive interface is a film with adhesive applied thereto that forms an adhesive border over the dressing leaving an open area.

In one embodiment of the invention, the diffusion dressing of claim 13 further includes an absorption layer and wherein the absorption layer lies between the permeable layer and the cellular layer. In another embodiment, the diffusion dressing of claim 13 further includes an absorption layer that also functions as the permeable layer, the absorbent layer being one of a hydrocolloid layer, a layer of beads, a layer of gel or a layer of material with or without perforations. In one embodiment of the diffusion dressing of claim 13, the adhesive interface is a film with adhesive applied thereto that forms an adhesive border over the dressing leaving an open area.

### BRIEF DESCRIPTION OF THE DRAWING FIGURES

FIG. 1 is a schematic diagram illustrating separate material layers and fabrication steps for combining the layers to make a diffusion dressing according to an embodiment of the present invention.

FIG. 2 is a cut view of a diffusion dressing fabricated from the material layers of FIG. 1.

FIG. 3 is a block diagram illustrating a convergence pattern of the separate material layers and fabrication steps for creating the diffusion dressing of FIG. 2.

FIG. 4 is a block diagram illustrating an overhead view of a diffusion dressing according to another embodiment of the present invention.

### DETAILED DESCRIPTION

The following detailed description of the invention refers to the accompanying drawings. The detailed description merely provides exemplary embodiment of the invention and is not intended to limit the invention.

The inventors provide a unique type of diffusion device that delivers a therapeutic agent such as gas to a target area such as tissue or cells and the novel manufacturing methods and processes to produce the therapeutic diffusion device. The device manufacturing of several device embodiments includes from a single to multiple reservoirs and materials containing the therapeutic agent.

The methods and apparatus of the present invention are described in enabling detail below using the following examples, which may include description of more than one embodiment of the present invention.

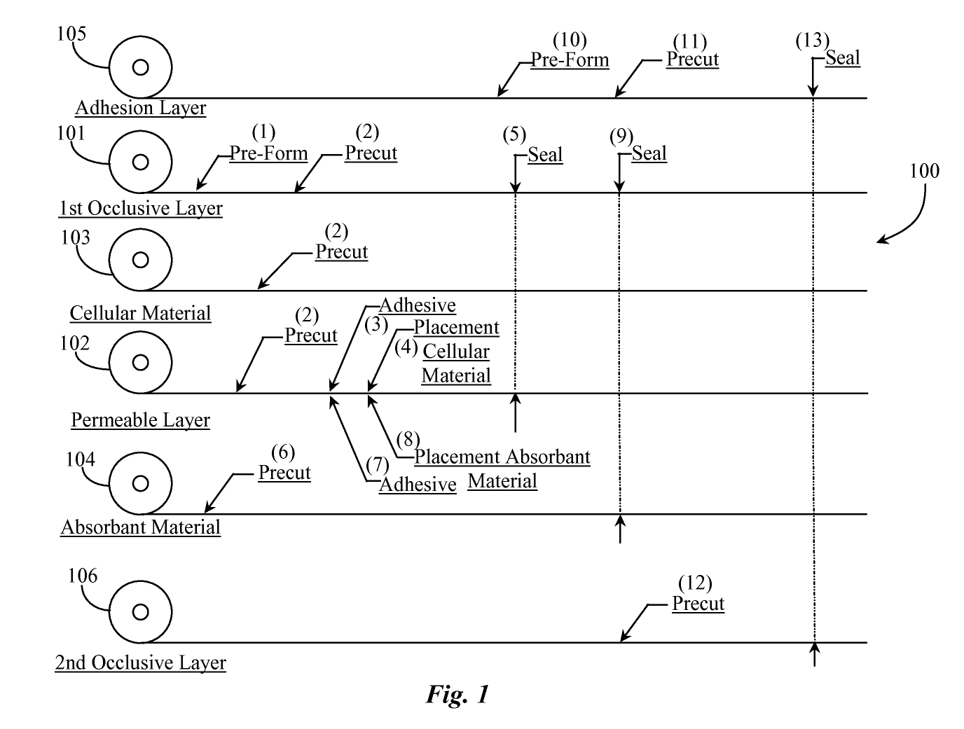

FIG. 1 is a schematic diagram 100 illustrating separate material layers and fabrication steps for combining the layers to make a diffusion device or a dressing according to an embodiment of the present invention. Diagram 100 logically represents several different material layers of a diffusion dressing and how they are integrated to produce a unique type of diffusion dressing in at least one embodiment of the present invention.

Diagram 100 logically represents several different material layers of a diffusion dressing and how they are integrated to produce a unique type of diffusion dressing in at least one embodiment of the present invention.

In this example there are six separate diffusion dressing material layers that are eventually integrated to form the diffusion dressing. The critical elements are a first occlusive material layer 101 adapted to provide a hermetic barrier to back-diffusion of therapeutic agent, a cellular material layer 103 adapted to contain and release at least one type of therapeutic agent through a permeable material layer 102 facing the tissue to be treated. Three additional material layers that may be considered optional layers in one embodiment include an absorbent material layer 104 adapted to absorb moisture and effluent from treated tissue, an adhesion layer 105 adapted to provide adhesion to the skin around the tissue treated, and a second occlusive material layer 106 adapted to function as a removable liner preserving the charged state of the diffusion dressing.

In this example, each material layer described is represented as a roll of material that can be fed into an automated process as required in an automated cutting, forming, and sealing system programmed to produce diffusion dressings comprising the material layers thus far described. Therefore, schematic diagram 100 describes a process of manufacture of a diffusion dressing inclusive of the described material layers. The order of materials from top to bottom is representative of the order of the layers as integrated into a diffusion dressing and not the order that might be staged for feed into an automated manufacturing system.

In one embodiment of the present invention the process calls for fabrication of the basic diffusion dressing unit comprising the three critical material layers 101, 102, and 103. First occlusive layer 101 may be a film stacked in a bin or wound about a roll as shown in this example. The material may be fed into a machine that pre-forms (1) the material layer. The pre-form application may be an inline system vacuum mold (not illustrated) using heat or cold vacuum forming process to garner a desired shape or indent of a specified geometric footprint and depth into the film. Typically the first occlusive layer fits over the reservoir material that contains the therapeutic agent. Pre-form depth of the material is typically about the thickness of cellular material 102 such that the film covers the top and sides of the material. First occlusive layer 101 is hermetic quality film in a preferred embodiment.

All three critical material layers 101, 102, and 103 are precut (2) to a finished size. Precutting may be accomplished using an inline rotary cutting die, a flatbed cutting die, or a hybrid machine incorporating both cutting die types. In one embodiment precutting may be accomplished using laser, electron beam cutting, or other cutting techniques. Cellular material 102 is precut to a finished size that is geometrically smaller (typically length and width) than the precut forms of the other two critical layers such that it may be encapsulated there between.

Cellular material 103 is, in a preferred embodiment, foam with an open cell structure promoting diffusion of therapeutic agent. In this example there is but one section of cellular material 103 per diffusion dressing. However, in some embodiments there are two or more discrete sections of cellular material provided in the footprint comprising the therapeutic agent reservoir, or in the case of multiple sections, reservoirs. In such an embodiment the cutting process may make more than one cut pattern in the material simultaneously. More detail about a unique diffusion dressing with multiple discreet reservoirs will be provided later in this specification.

Permeable layer 102 has an adhesive applied (3) to the footprint of the precut area so that the precut section of cellular material 103 may be placed (4) thereon using an island placement technique known to the inventor. Island placement is known in such as the processing of labels for example. Permeable layer 102 and first occlusive layer 101 are then sealed together (5) over each geometric footprint of a diffusion dressing encapsulating the cellular material (therapeutic reservoir) there between. This provides the basic component assembly of the diffusion dressing comprising a permeable bottom layer, a cellular reservoir, and an occlusive layer of hermetic quality.

Absorbent layer 104 may be an open cell layer, polyurethane foam layer, a non-woven rayon layer, a layer of blown microfibers, a hydrocolloid layer, a layer of beads, a layer of gel or a layer of some other absorbent material with or without perforations. Absorbent material 104 is optional and may be used to absorb moister and effluent from the tissue being treated. Absorbent layer 104 is precut (6) into a geometric footprint similar to that of permeable layer 102 for a single diffusion dressing. It is noted herein that in one embodiment precutting does not part off the pieces or sections of material. Rather, as the component is layered, it remains a finished roll of diffusion dressing that may be separated in terms of individual devices by tearing at a perforation line.

Permeable layer 102 may receive adhesive (7) on the underside at the geometric footprint location to enable island placement (8) of a section of absorbent material directly beneath the therapeutic reservoir. In one embodiment, all of the precutting is done at the same time and layers converge as adhesive and island placement occurs creating a roll of diffusion dressings or multiple dressings that are parted off at the end of the manufacturing process.

In one embodiment of the present invention a thin non-adherent film can be attached to the underside of absorbent material layer 104 (side opposite of therapeutic agent reservoir) to prevent tissue growth into the absorbent layer. In another embodiment, adhesive (7) might be applied to the surface of absorbent material 104 facing the therapeutic reservoir instead of to the underside of the permeable layer to facilitate island placement of the absorbent material to the permeable layer. It is also noted herein that adhesive used in between material layers may be permeable where required. Sealing of the first occlusive layer to the permeable layer may be accomplished using any of known sealing techniques including but not limiting to heat sealing, radio frequency (RF) cold welding, electron beam welding, pressure sensitive adhesion, or like methods.

Absorbent material layer 104 is sealed (9) against first occlusive layer 101 using an impermeable gel (hardening or non-hardening), the gel impermeable to therapeutic agent. The gel creates a peripheral seal around the diffusion dressing. Seal (9) can be a double-sided occlusive tape that can overlap absorbent layer 104 on either side at its periphery. Adhesion layer 105 fits over the top of the diffusion dressing and provides adhesion against the skin of a user local to the treated wound. Adhesion layer 105 is pre-formed in a similar fashion as described above with respect to the first occlusive layer. Pre-form techniques may include vacuum molding using a cold or heat vacuum process. After pre-forming (10) layer 105 is precut (11) to a finished size.

2nd occlusive layer 106 may be a continuous film of hermetic quality similar to the 1^{st} occlusive layer. Occlusive layer 106 is a protective liner that must be removed before applying the diffusing dressing to the skin of a user. Eventually, layer 106 is sealed (13) after precutting (12) to adhesion layer 105 to finish diffusion dressing 100. Adhesion layer 105 may cover the entire dressing or may just cover the periphery of the dressing. Adhesion layer 105 provides the ability to stick to a user's skin during application keeping the diffusion dressing secure over the wound.

In one embodiment the permeable layer is a liner with small perforations provided therethrough and symmetrically arranged in a symmetrical pattern. In this embodiment an absorbent layer may be a hydrocolloid layer with larger perorations held in roughly the same pattern as the smaller perforations in the liner.

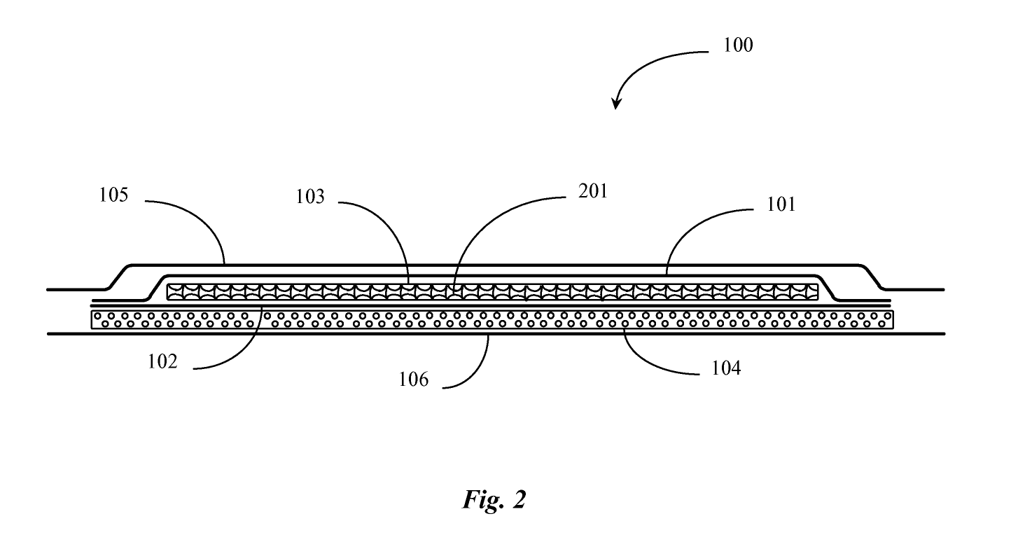

FIG. 2 is a cut view of diffusion dressing 100 fabricated from the material layers of FIG. 1. Diffusion dressing 100 exhibits the six material layers illustrated previously in FIG. 1. Diffusion dressing 100 includes material layers 101, 102, and 103 making up the core diffusion dressing. Absorbent layer 104 located beneath permeable layer 102 is sealed to the permeable layer using occlusive double-sided tape or some other sealing method such as a bead of impermeable gel.

Adhesion layer 105 and the second occlusive layer 104 are sealed to one another such that the occlusive liner at the bottom prevents diffusion of the therapeutic agent or agents contained within cellular material layer 103. In one embodiment further layers are added to package the diffusion device for shipment. However, the two occlusive layers of hermetic quality provide a seal sufficient to underscore any packaging. In one embodiment, finished diffusion dressings like dressing 100 are parted off when they come to the end of the conveyor of the machine hosting the automated process.

In another embodiment, the diffusion dressings are collected in a roll of finished product that may then be shipped to an ordering party. In one embodiment the process of the present invention may be altered somewhat for different dressing architectures. For example, absorbent layer 104 is no absolutely required and is an optional component for use in collecting effluent from a wound. In one embodiment cellular layer 103 is pre-charged with therapeutic agent before assembly of the complete diffusion dressing is completed. In another embodiment, the cellular material is integrated into the diffusion dressing dry and then charged at or near the end of assembly to minimize the amount of therapeutic agent escaping from or diffusing from the dressing before final sealing of the liner (106) to the adhesion layer (105).

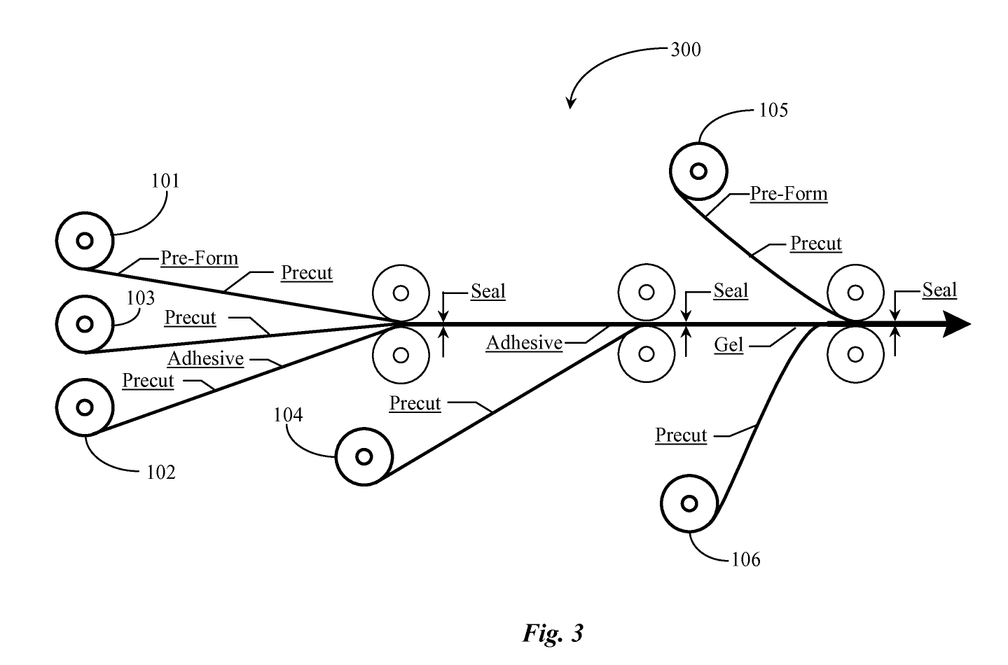

FIG. 3 is a block diagram 300 illustrating a convergence pattern of the separate material layers and fabrication steps for creating the diffusion dressing of FIG. 2. In one embodiment of the present invention, the critical component layers of the diffusion dressing are assembled first using the automated process of the present invention. First occlusive layer 101, cellular material layer 103, and permeable layer 102 may be staged and assembled first in one embodiment resulting in a single diffusion dressing layer including the basic and critical components after sealing.

The preparation tasks such as pre-forming and precutting may staged as illustrated, for example, performing the first occlusive layer first followed by the precutting task for all three components. Application of adhesive may be performed just before island placement of cellular material 103 onto the permeable layer 102. Sealing is between the first occlusive layer and the permeable layer and may occur after the layers are brought close enough together for the sealing process. It is important to note that material layer backing might be provided to keep cut pieces from falling away, such material being peeled away as the layers are integrated leaving the precut components in the remaining layer sealed in appropriate fashion. The components may remain connected together via perforated line separating one dressing from a next dressing.

After the first three basic layers are assembled, absorbent material 104 might be added by precutting the absorbent material pieces, applying adhesive to the remaining side of layer 102. The absorbent layer is sealed to the underside of the diffusion “core”.

Adhesion layer 105 and the second occlusive layer or (liner) 106 may be the last components integrated into the diffusion dressing. These components are incorporated into the dressing and sealed to form a single unbroken material layer comprising a line of finished diffusion dressing that may be parted off at the end of manufacture or that may be incorporated onto a finished role of product that may be shipped out to a client. In one embodiment, a bead of impermeable gel is applied to affect a seal between the adhesion layer 105 and the liner 106. After final sealing, a single material layer containing completed diffusion dressings may be output from the machine hosting manufacture of the product.

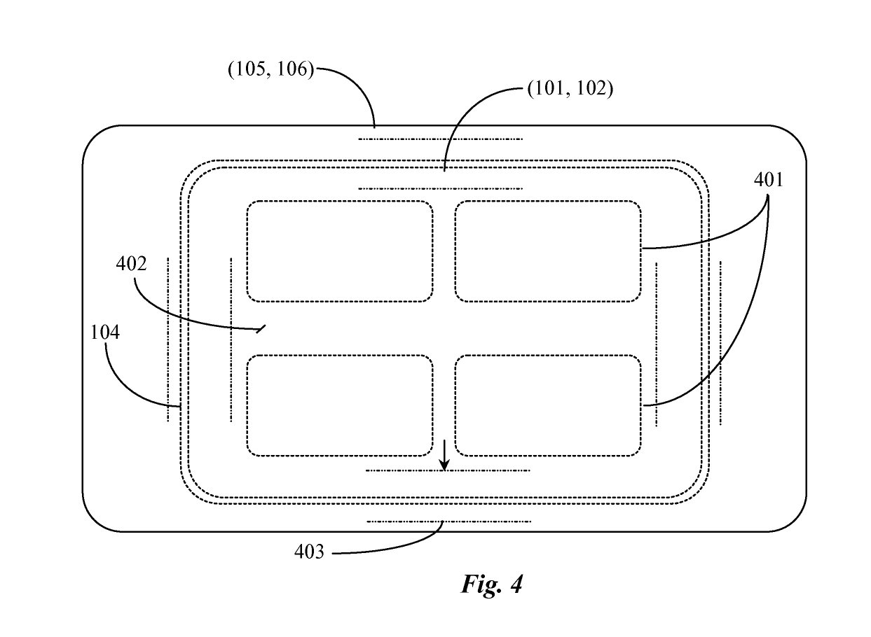

FIG. 4 is a block diagram illustrating an overhead view of a diffusion dressing 400 according to another embodiment of the present invention. In this example, diffusion dressing 400 incorporates adhesion layer 105 and the second occlusion layer 106 sealed together. The first occlusive layer 101 and permeable layer 102 enclose multiple discrete or separate cellular material sections 401 that may hold the same or varied therapeutic agents. In one embodiment first occlusive layer 101 is sealed to permeable layer 102 around the multiple cellular sections using a press sealing technique resulting in a hermetic seal 402.

In this example there are four material sections 401 but there may be more or fewer sections provided in a dressing without departing from the spirit and scope of the present invention. Material layer 104 is just larger in overall dimensioning that the critical first occlusive and permeable layers. A double-sided occlusive film or tape 403 (broken parallel lines) may be used to seal absorbent layer 104 to diffusion device 400 in much the same manner as previously described for a dressing containing only one therapeutic reservoir.

In one embodiment cellular material reservoirs 401 have different therapeutic agents infused therein and may have diffusion rates of diffusion as well. In one example, instead of the second occlusive layer 106 serving as a liner, there are several liners, one per reservoir. In this way, one or more liners may be removed to diffuse the associated therapeutic agents or agent into the tissue wound while the remaining therapeutic reservoirs are reserved for activation later in the healing process.

In one embodiment different diffusion rates are ordered and controlled by thickness dimensioning of permeable adhesive applied to seal a “liner” to a “reservoir”. For example, if a lower diffusion rate is desired for a reservoir, then more adhesive is applied slowing the rate of diffusion for that reservoir. If a high rate of diffusion is desired for a specific therapeutic agent, then a very thin application of permeable adhesive might be applied to maximize the rate for that reservoir.

In one embodiment different therapeutic reservoirs placed on the permeable layer are charged just before sealing by injection or infusion of the agent into the cellular material defining a reservoir. In one embodiment of the invention one or more charge ports might be provided for charging therapeutic reservoirs on a diffusion dressing. In another embodiment, the reservoir sections might be charged before island placement of the sections onto the permeable layer of the diffusion dressing. There may be many different configurations without departing from the spirit and scope of the present invention. Such different configurations might depend upon the different natures of the wounds to be treated with the specific dressings. For example, a burn might be treated differently that a puncture wound and may need a different dressing than one required for a puncture wound. A machine hosting the assembly or manufacture process might be re-tooled to manufacture different types of dressings.

It will be apparent to one with skill in the art that the diffusion dressing and manufacturing process of the invention may be provided using some or all of the mentioned features and components without departing from the spirit and scope of the present invention. It will also be apparent to the skilled artisan that the embodiments described above are specific examples of a single broader invention which may have greater scope than any of the singular descriptions taught. There may be many alterations made in the descriptions without departing from the spirit and scope of the present invention.

What is claimed is:

1. A method for manufacturing a diffusion device comprising the steps:
(a) pre-forming and precutting a first occlusive layer of material to a finished size;
(b) precutting a permeable layer of material to a finished size;
(c) coating one side of the permeable layer of step (b) with a permeable adhesive;
(d) placing one or more precut sections of a cellular material layer onto the adhesive side of the permeable layer of step (b);
(e) sealing the first occlusive layer of step (a) to the permeable layer of step (b) encapsulating the one or more sections of cellular material of step (d);
(f) precutting a section of absorbent material to a finished size;
(g) coating the remaining side of the permeable layer of step (b) with a permeable adhesive;
(h) placing the section of absorbent material on the coated side of the permeable layer of step (g);
(i) applying a seal about the periphery of the remaining side of the absorbent material forming a peripheral seal to the occlusive layer;
(j) pre-forming and precutting a breathable adhesion layer to a finished size sufficient to cover at least the peripheral area of the diffusion device thus far assembled;
(k) precutting a removable second occlusive layer of material to a finished size; and
(l) sealing the second occlusive layer against the adhesion layer of step (j).

2. The method of claim 1 wherein in step (a) and in step (j) pre-forming is accomplished using a cold vacuum or hot vacuum forming tool.

3. The method of claim 1 wherein in steps (a), (b), (f), (j), and (k), precutting is accomplished using a flatbed, rotary, or hybrid die cutting tool.

4. The method of claim 1 wherein in step (d) and in step (h) the material sections are placed via an island placement technique.

5. The method of claim 1 wherein in step (a) and step (k) the occlusive layers are continuous hermetic quality films.

6. The method of claim 1 wherein in step (d) the cellular material is foam having an open cell structure.

7. The method of claim 1 wherein in step (f) the absorbent material is a hydrocolloid, and or one of open cell polyurethane foam, a non-woven rayon, blown microfibers, absorbent beads, adhesive material or gel.

8. The method of claim 1 wherein in steps (e), (i), and (l), sealing is accomplished using one of heat sealing, radio frequency (RF) cold welding, electron beam welding, or pressure sensitive adhesive.

9. The method of claim 1 wherein in step (i) sealing is accomplished using a double-sided occlusive tape or by laying a bead of impermeable gel.

10. The method of claim 1 wherein an additional step is provided between step (h) and step (i) for attaching a non-adhesive film to the remaining side of the absorbent layer to prevent tissue from growing into the absorbent layer.

11. The method of claim 1 wherein in an additional step is provided between step (h) and step (i) for applying a gel, a cream, or any other compound that is bio-absorbable or bio-degradable.

12. The method of claim 11 wherein the compound is inert or contains one or more drugs such as an anesthetic, an analgesic or a combination of therapeutic agents.

13. A diffusion dressing comprising:
two or more discrete sections of cellular material enclosed by an occlusive material layer and a permeable material layer; and
a sealable adhesive interface for maintaining the dressing in a state of adhesion against a tissue area being treated.

14. The diffusion dressing of claim 13 wherein the cellular material is foam having an open cell structure.

15. The diffusion dressing of claim 13 wherein the two or more discrete sections of cellular material are charged with a same therapeutic agent.

16. The diffusion dressing of claim 13 wherein the two or more discrete sections of cellular material are individually charged with different therapeutic agents.

17. The diffusing dressing of claim 15 wherein the two or more discrete sections of cellular material have different rates of diffusion.

18. The diffusion dressing of claim 15 wherein the rate of diffusion of each section of material is controlled by controlling the thickness of and or number of layers of permeable adhesive added to the diffusion membrane directly beneath a cellular material section.

19. The diffusion dressing of claim 13 wherein the sealable interface comprises an adhesion layer and a second occlusive layer.

20. The diffusion dressing of claim 19 wherein the adhesion layer is a breathable skin adhesive layer and the second occlusive layer is a removable liner that is removed before application of the diffusion dressing.

21. The diffusion dressing of claim 13 further including an absorption layer and wherein the absorption layer lies between the permeable layer and the cellular layer.

22. The diffusion dressing of claim 13 further including an absorption layer that also functions as the permeable layer, the absorbent layer being one of a hydrocolloid layer, a layer of beads, a layer of gel or a layer of material with or without perforations.

23. The diffusion dressing of claim 13 wherein the adhesive interface is a film with adhesive applied thereto that forms an adhesive border over the dressing leaving an open area.
